(19) United States
(12) Patent Application Publication
(10) Pub. No.: US 2014/0350660 A1
(43) Pub. Date: Nov. 27, 2014

(54) Endoluminal Prosthesis

(71) Applicants: Graeme Cocks, Crows Nest (AU); Gary S. Roubin, New York, NY (US); Ronald John Laidlaw Dick, Brighton (AU); Kathleen White, Birchgrove (AU); Zoran Milijasevic, Bayview (AU)

(72) Inventors: Graeme Cocks, Crows Nest (AU); Gary S. Roubin, New York, NY (US); Ronald John Laidlaw Dick, Brighton (AU); Kathleen White, Birchgrove (AU); Zoran Milijasevic, Bayview (AU)

(21) Appl. No.: 14/361,395

(22) PCT Filed: Nov. 14, 2012
(86) PCT No.: PCT/AU2012/001402

§ 371 Date: May 29, 2014

(30) Foreign Application Priority Data
Dec. 1, 2011 (AU) 2011905007
Aug. 1, 2012 (AU) 2012903302

(51) Int. Cl. A61F 2/24; A61F 2/82

(52) U.S. Cl. CPC A61F 2/2418; A61F 2/82; A61F 2220/0075; A61F 2250/0018; A61F 2250/0037
USPC 623/1.16

(57) ABSTRACT

An endoluminal prosthesis for deployment in a body which has a first tubular structure for stabilising the prosthesis, the first tubular structure being substantially concentric about a first axis and movable between a compact and an expanded state. The prosthesis also has a second tubular structure for supporting an artificial heart valve, the second tubular structure being substantially concentric about a second axis and moveable between a compact and an expanded state. The first axis and the second axis extend at different angles when the first and second structures are in the compact and/or expanded state. A third structure which is shape and/or length adjustable is positioned between the first and second structures.

### FIELD

The present disclosure relates to an endoluminal prosthesis for use in the vasculature and/or heart including, but not necessarily limited to, a prosthesis for replacing or repairing a damaged aortic valve.

### BACKGROUND

Endoluminal prostheses including stents are normally used to keep blood vessels open or “patent”. Commonly, stents are single component cylindrical cages that can be collapsed and introduced into a blood vessel before being expanded into place. Stents are normally either self expanding or balloon expandable. Self expanding stents are usually made from a memory material that springs open towards a predetermined size when released from an introducing catheter. Balloon expanding stents are usually made from an annealed type of material, with a balloon being inflated to expand the stent and deflated to allow removal of the balloon. The stent remains at the diameter that the balloon expands it to less any back contraction caused by the vessel walls.

An endoluminal prosthesis is usually deployed by advancing the prosthesis along a delivery catheter through a relatively small incision in a blood vessel. Due to the minimally invasive nature of stents, they are popular with both surgeons and patients. For this reason also, prostheses commonly comprise devices that are supported by one or more stents, including replacement heart valves.

To increase the stability of the heart valve it is known to modify a supporting stent so that it is relatively longer and has two regions of different diameters for placement in respective parts of the vascular system. The anatomy around heart valves such as the aortic valve is variable, particularly with respect to curvatures, angulations and dimensions. It has therefore been found that prostheses are difficult to deploy correctly at this region, with regurgitation being a common problem. Further, the strength of the stent may be varied at different regions of conventional stents, several devices currently in use employing high radial strength in the lowest most region of the stent which anchors the stent valve in place at and below the native annulus. The problem with such high radial force at this region however is that damage may be caused to surrounding structures and in particular the conductive tissue of the heart, for example at the Bundle of His. If the force of the stent valve damages this region, it is necessary to implant a pacemaker to artificially pace the heart in place of natural pacing.

There is clearly a need for an easy to deploy device that allows accurate positioning within a region of the heart and which provides optimal anchoring whilst avoiding any damage to the pacing/conductive fibres of the heart.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

In one aspect, the present disclosure provides an endoluminal prosthesis for deployment in a body, comprising:

a first expandable structure,

a second expandable structure; and

a third structure connected between the first and second structures,

the third structure having an adjustable shape such as to permit displacement of one of the first and second structures relative to the body independently of displacement of the other of the first and second structures relative to the body.

In a second aspect, there is provided an endoluminal prosthesis for deployment in a body, said prosthesis having a superior end region and a posterior end region and comprising:

a first expandable structure,

a second expandable structure;

a third structure between the first and second structures, and

a fourth expandable structure extending posteriorly from said second structure; said endoluminal prosthesis configured to be radially expandable from an initial compact state to an expanded state and, when the prosthesis is in its expanded state, each structure has a radial strength, and wherein said fourth structure has relatively less radial strength than at least part of said first structure.

The endoluminal prosthesis is typically configured for deployment in the vasculature and/or heart. The first structure and/or second structure may be configured to be expandable from a compact state for delivery to an expanded state where it contacts an internal surface of the vasculature and/or heart. The first and/or second structure may be configured to support a prosthetic device. For example, the prosthetic device may be an artificial valve configured to replace or repair a heart valve such as the aortic valve. The second structure may support the heart valve and the first structure may provide for stabilisation of the second structure, for example.

In one embodiment, when the prosthesis is deployed, the first structure is configured to locate at least partially in the ascending aorta, the second structure is configured to support the artificial valve and locate at least partially across the native aortic valve, and the third structure is configured to locate at or adjacent the sinotubular junction of the aorta where the right and left coronary arteries meet the aorta.

At least one of the first and second structures, e.g., the second structure, may support a biocompatible graft material attached to the artificial valve and the graft material may be configured to seal against the aortic wall.

The first and second structures may each be substantially tubular, allowing blood to flow therethrough. The first and second structures may be substantially cylindrical. The first and second structures may be respective stents, e.g., balloon expandable and/or self-expanding stents. For example, the first structure may be a self-expanding stent and the second structure may be a balloon-expandable stent. Stents having any shape suitable for expansion to fit the respective anatomical spaces may be used. When fully expanded, the first structure may have a diameter greater than the second structure.

The third structure may be substantially tubular to allow blood flow therethrough. The third structure may be substantially cylindrical. The third structure may have an adjustable shape or position relative to the first and/or second structures. However, due to the shape-adjustable nature of the third structure, it may take a variety of different shapes, whether naturally (e.g. in a fully expanded state), or as a result of forces imparted on it via the first and second structures and/or imparted on it by vessel walls. For example, although the first and second structures may each be substantially cylindrical, the first and second structures may have different diameters, as indicated above, and therefore the third structure may have a larger diameter at one end and a smaller diameter at the opposite end. The third structure may therefore have a substantially frusto-conical shape. Furthermore, the first and second structures may be aligned eccentrically (i.e. non-concentrically), and therefore the third structure may have an asymmetric form. The third structure may be self-expandable or balloon expandable. The third structure may be expanded partially or entirely as a consequence of self expansion and/or balloon expansion of the first and second structures or may be inherently self-expandable or configured to be expanded directly using a balloon.

The shape-adjustability can be present before, during and/or after the third structure has expanded within the blood vessel. The third structure may be shape-adjustable through having one or more flexible and/or relatively movable parts. These parts may generally define the outer shape of the third structure, leaving a central region of the third structure open for blood flow. The shape of the third structure may be more easily adjustable than the shape of the first and/or second structures. By configuring the third structure to be shape-adjustable, in a manner that permits displacement of one of the first and second structures relative to the body independently of displacement of the other of the first and second structures relative to the body, the orientation and positioning of one of the first and second structures relative to the body can be adjusted without affecting the orientation or positioning of the other of the first and second structures relative to the body, allowing the most desirable position and orientation of the first and second structures to be achieved for both anchoring the prosthesis, and locating of the artificial heart valve, respectively, for example. This may prevent regurgitation as a result of blood flowing back past the second structure through displacement of the second structure that might, otherwise be caused by displacement of the first structure.

The prosthesis will generally include an axis extending from a distal to a proximal end of the prosthesis through the first, second and third structures (hereinafter “the main axis”), the main axis aligning substantially with the flow path of the aortic valve when the prosthesis is deployed. In certain embodiments, e.g. prior to deployment, the first, second and third structures may be concentric about the main axis when the prosthesis is in its fully expanded state. In alternative embodiments, one or more of the first, second and third structures may be eccentrically aligned with the main axis, such that the prosthesis has a bent or curved shape, e.g. in its fully expanded state.

The eccentric alignment may more closely follow the anatomy of the aortic root and valve, where an eccentric alignment between the ascending aorta and the aortic channel below the sinotubular junction is common. Thus, the prosthesis may have a preformed eccentric configuration, with the shape adjustability of the third structure allowing relative independent movement of the first and second structures from an eccentrically aligned starting point of the first and second structures.

Once the first and second structures are positioned appropriately for use, the shape of the third structure may be fixed or locked such as to ensure a firm connection between the first and second structures via the third structure. This may ensure that the first structure provides sufficient stabilisation of the second structure for reliable functioning of e.g. the artificial heart valve. As an alternative, the shape of the third structure may remain shape-adjustable, e.g., to a limited extent, ensuring that relative movement of the first and second structures remains possible during initial or continued use of the prosthesis. This may allow for adaptation, of the shape of the prosthesis to movements of the vascular system. In one embodiment, the third structure may be configured such that the relative movement of the first and second structures may be limited to a greater degree along one axis of the prosthesis in comparison to a perpendicular axis. For example, the relative movement of the first and second structures may be more limited along the main axis, and therefore also along the flow path of the aortic valve, than in a lateral direction substantially perpendicular to the main axis, or vice-versa.

The shape adaptability of the third structure may be such as to allow the first and second structures to move towards or away from each other, translate with respect to each other and/or relatively rotate with respect to each other. The movement towards or away from each may be in a direction parallel to the main axis and the translation and/or relative rotation may be within one or more planes parallel to the main axis, for example.

The shape adaptability of the third structure may be achieved in a variety of different ways. As one example, the third structure may comprise a plurality of connectors connected between the first and second structures, wherein the lengths of the connectors between the first and second structures are adjustable with respect to each other. The connectors may define the shape of the third structure.

In a third aspect, the present disclosure provides an endoluminal prosthesis for deployment in a body, comprising:

a first expandable structure,

a second expandable structure; and

a third structure comprising a plurality of connectors connected between the first and second structures,

wherein the lengths of the connectors between the first and second structures are relatively adjustable such as to permit displacement of one of the first and second structures relative to the body independently of displacement of the other of the first and second structures relative to the body.

References herein to the length of connectors between the first and second structures are intended to describe a straight-line distance between connection points of the connectors and the first and second structures, respectively. Accordingly, the length of the connectors between the first and second structures may be shorter than a path (e.g. a curved or bent path) that the connectors follow between the first and second structures.

In any of the aspects disclosed herein, the lengths of the connectors between the first and second structures may be relatively adjustable substantially independently of each other, such that variation of the length of one connector does not necessarily affect the length of another connector. By relatively adjusting the lengths of the connectors between the first and second structures, the first and second structures may be translated or relatively rotated as indicated above. For example, if the lengths of the connectors between the first and second structures on one side of the prosthesis are decreased, the first and second structures will move closer to each other on that side of the prosthesis, causing relative rotation of the first and second structures. The relative movement may be sufficient to better align the first and second structures in their respective desirable deployment positions. Although the lengths of connectors may be relatively variable, the lengths may also be variable in unison, i.e. to the same degree at the same time, permitting the first and second structures to move in a linear manner towards and away from each other. The connectors may be narrow, elongate members, with first and second ends of the connectors being connected directly to the first and second structures respectively, or connected to the first and second structures via additional parts of the third structure, e.g. via a connecting element at one or both ends of the third structure. The connectors may take a variety of different forms. For example, the connectors may be straight or curved or bent and may be one-piece or comprise multiple pieces. Any element capable of varying its dimension between the first and second structures may be a type of connector suitable for the purposes of the present disclosure. Therefore, although, for simplicity, the term “length” has been used to describe straight-line distances traveled by the connectors between the first and second structures, the direction of elongation of the connectors need not necessarily extend in this length direction in all embodiments.

To achieve the length variation between the first and second structures, the connectors may, for example, comprise first and second portions that are connected to the first and second structures, respectively, wherein the first and second portions are relatively moveable. For example, the first and second portions may be slidably connected. By sliding the first and second portions relative to each other, the length of the connector between the first and second structures may be varied. As an alternative, the connector may be a single piece or otherwise and may be slidably connected to the first and/or second structures. By sliding the connector relative to the first and/or second structures, the length of the part of the connector that bridges the gap between the first and second structures is varied, meaning in turn that the length of the connector between the first and second structures is varied. As another example, first and second portions may be provided that are pivotably connected to each other (e.g. via a hinge), such that relative pivoting of the first and second portions causes the length of the connector between the first and second structures to be varied.

As exemplary alternatives to having distinct relatively moveable portions as described above, the connectors may be one-piece or otherwise and be flexible and shaped in a manner that allows the lengths of the connectors between the first and second structures to be varied. For example, one or more of the connectors may comprise a curved and/or bent region and, due to the flexibility of the connectors, the degree of curvature and/or bending of the curved and/or bent region may be adjustable to vary the length of the connector between the first and second structures. The curved and/or bent region may be at least partially s-shaped, zigzag-shaped and/or folded or otherwise.

The connectors may be connected to the first and/or second structures via respective flexible or articulating joints. The joints may allow rotation of the connectors relative to the first and/or second structures, e.g. upon varying the lengths of different connectors as described above.

The connectors may be separate from each other. Alternatively, one or more of the connectors may be interconnected, e.g. via hinges or flexible connections.

In any of the aspects described herein, to fix or lock the shape of the third structure, a locking element may be provided that engages one or more of the connectors in a manner that restricts the length variability of the connectors between the first and structure structures and/or rotation of the connectors at the joints. Alternatively, the connectors or joints may be deformed to achieve the same effect. In one example, an expandable stent may be provided as a locking element, wherein, upon expansion, the stent engages the connectors and prevents any further movement of all or part of the connectors. In one example, where a connector comprises first and second relatively slidable portions, to fix the length of the connector between the first and second structures, one or both of the first and second portions may be deformed. For example one or both of the first and second portions may be deformed beyond an elastic limit, such that relative sliding via the slidable mounting is no longer possible. For example, if the first portion is slidably mounted to the second portion by slidably extending through an aperture connected to or integral with the second portion, the first portion may be bent or kinked in a manner that obstructs sliding of the first portion through the aperture. Deformation may be achieved by expanding a balloon and/or expanding a stent. When fixed or locked, the third structure may or may not retain a level of flexibility. Any flexibility that is maintained may not be such as to permit independent movement of the first and second structures, as might otherwise be possible prior to fixing or locking.

As an alternative to the first and second structures being substantially cylindrical, the first structure and/or second structures may have curved sides. For example, the first and/or second structures may be barrel-shaped, with convex outer side surfaces, such that the diameter of the first structure and/or second structure is generally greater towards a central region than at its ends. By designing one or both of the first and second structures in this manner, the structures may have the ability to self align within the vessel, and/or allow easier repositioning. Curved sides may also provide the structures with greater strength to counteract compressive forces, in comparison to substantially straight sides.

A belt having an outer surface with a relatively low coefficient of friction, e.g. a belt comprising Teflon™ or polyethylene, may be provided around the middle portion of the barrel shaped structures allowing small amounts of rotation for self alignment. The rotation may be partially limited by the thickness of the belt.

Although configurations in which three structures are provided have been described above, the prosthesis may include additional or fewer structures. For example, the first structure may comprise a plurality of different elements, e.g. multiple stents, which are connected together (daisy chained).

The third structure may be split into at least two components which may allow limited movements in one direction and completely different movement or lack of movement in another direction, e.g. in a direction perpendicular to the first direction.

The first, second and/or third structures of the prosthesis may be formed at least in part from Nitinol™, stainless steel, or elgiloy. Examples of other materials that may be used to form any one or more of the structures include: carbon, carbon fiber, chromium, cobalt, gold, inconel, iridium, platinum, silver, tantalum, titanium, tungsten, cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers thereof; polylactic acid, polyglycolic acid or copolymers thereof; a polyanhydride, polycaprolactone, or polyhydroxybutyrate valerate. However, other biocompatible metals, alloys, or biodegradable polymers or mixtures or copolymers may be used.

Where graft material is used in conjunction with the second structure or otherwise, the graft material may be formed of polyethylene terephthalate polytetrafluoroethylene (PTFE), expanded PTFE, and other synthetic materials, or naturally occurring biomaterials such as collagen, and may have a size according to the dimensions of the artery under treatment or have a predetermined size, such as a size that exceeds most diameters of recipient arteries.

One of more of the first, second and third structures may be fitted with sealing rings, located on an outer surface thereof. For example, the second structure may be fitted with sealing rings made from e.g., silicone, rubber or soft plastic. Between the sealing rings there may be a polyester mesh or similar material that can coagulate with the presence of blood and may prevent backflow of blood and aid in anchoring of the prosthesis.

In the aspects described above, the third structure is described as having an adjustable shape and/or having connectors of adjustable length such as to permit displacement of one of the first and second structures relative to the body independently of displacement of the other of the first and second structures relative to the body. Although this independent movement is desirable, in alternative aspects, features of the first and second aspects may be employed without necessarily requiring that the first and second structures are independently moveable relative to the body. Despite this, the alternative aspects may still employ features, including features that are described with respect to the above aspects, which are advantageous in aligning the first and second structures of the prosthesis in respective deployment positions.

For example, in accordance with a fourth aspect, there is provided an endoluminal prosthesis for deployment in a body, comprising:

a first expandable structure,

a second expandable structure; and

a third structure connected between the first and second structures,

wherein the third structure comprises a plurality of connectors connected between the first and second structures, the lengths of the connectors between the first and second structures being adjustable relative to each other.

In accordance with a fifth aspect, there is provided an endoluminal prosthesis for deployment in a body, comprising:

a first expandable structure,

a second expandable structure; and

a third structure connected between the first and second structures,

wherein the third structure comprises a plurality of connectors, each connector having at least a first portion connected to the first structure and a second portion connected to the second structure, the first portion being slidably mounted to the second portion such that the length of the connector between the first and second structures is adjustable by sliding of the first portion relative to the second portion.

In accordance with a sixth aspect, there is provided an endoluminal prosthesis for deployment in a body, comprising:

a first expandable structure,

a second expandable structure; and

a third structure connected between the first and second structures,

wherein the third structure comprises a plurality of connectors, each connector being slidably mounted to the first structure and/or the second structure such that the length of the connector between the first and second structures is adjustable by sliding of the connector relative to the first and/or second structure.

In accordance with a seventh aspect, there is provided an endoluminal prosthesis for deployment in a body, comprising:

a first expandable structure,

a second expandable structure; and

a third structure connected between the first and second structures,

wherein the third structure comprises a plurality of connectors, each connector comprising a curved and/or bent portion, wherein the length of each connector between the first and second structures is adjustable by adjusting a degree of curvature and/or bending of the connector.

In accordance with a eighth aspect, there is provided an endoluminal prosthesis for deployment in a body, comprising:

a first expandable structure,

a second expandable structure; and

a third structure connected between the first and second structures,

wherein the third structure comprises a plurality of connectors, each connector being connected to the first and/or second structure via an articulating joint.

In accordance with an ninth aspect, there is provided an endoluminal prosthesis for deployment in a body, comprising:

a first expandable structure,

a second expandable structure; and

a third structure connected between the first and second structures,

wherein one or both of the first and second structures has convex outer side surfaces.

It will be clear to the skilled person that various features of the prosthesis of the different aspects described above, which have first, second and third structures, may be combined. In general, the features of the prosthesis according to any one of these aspects may be as described with respect to any other of the aspects, and the prosthesis according to any one of the aspects may include additional features described with respect to any other of the other aspects.

As discussed above, the first and second structures of the prosthesis may have an eccentric alignment such as to more closely follow the anatomy of the aortic root and valve, where an eccentric alignment between the ascending aorta and the aortic channel below the sinotubular junction is common. The prosthesis may have a preformed eccentric configuration. Accordingly, even in a compact form for delivery, an angulation between the first and second structures may be present. In certain preceding aspects, shape adjustability of a third structure is described to allow relative movement of the first and second structures from an eccentrically aligned starting point of the first and second structures. However, in alternative aspects, the angulation may substantially obviate the requirement for any substantial relative movement of the first and second structures, by naturally conforming the prosthesis to the anatomy of the aortic root and valve. An expandable third structure between the first and second structures may therefore be provided having a substantially fixed configuration (save for its expandability), such as to maintain a desired eccentric alignment of the first and second structures.

In accordance with a tenth aspect, there is provided an endoluminal prosthesis for deployment in a body, comprising:

a first expandable tubular structure for stabilising the prosthesis, and

a second expandable tubular structure for supporting an artificial heart valve;

wherein the first and second structures are eccentrically aligned when in a compacted and/or expanded state.

The eccentric alignment may be such that the first and second structures are substantially concentric about first and second axes, respectively, wherein the first and second axes may be substantially parallel but not collinear, or wherein the first and second axes extend at different angles.

In accordance with an eleventh aspect, there is provided an endoluminal prosthesis for deployment in a body, comprising:

a first tubular structure for stabilising the prosthesis, the first tubular structure being substantially concentric about a first axis and movable between a compact and an expanded state, and

a second tubular structure for supporting an artificial heart valve, the second tubular structure being substantially concentric about a second axis and moveable between a compact and an expanded state;

wherein the first axis and the second axis extend at different angles when the first and second structures are in the compact and/or expanded state.

The angle between the first and second axes may be from 5 degrees to 30 degrees, or from 10 to 20 degrees, for example, or otherwise.

As indicated, the prosthesis may include a third structure located between the first and second structures. The third structure may have adjustable shape and/or include length adjustable connectors as described with respect to preceding aspects. Alternatively, the structure may have a substantially fixed configuration such as to maintain the desired angle between the first and second structures both during delivery to the deployment site and after expansion of the first and second structures.

The eccentric/angulated alignment between the first and second structures may be achieved through using a shape-memory material such as Nitinol™ in the prosthesis, e.g. in the third structure of the prosthesis; or by physical angulation of a malleable metal of the prosthesis in the manufacture stage or post manufacture e.g. with the prosthesis in a compressed state (e.g. prior to packaging in a delivery sheath, or even at the time of the deployment procedure); or through use of different lengths of connectors in the third structure; or by other means.

The prosthesis according to any one of the aspects may be configured to be delivered, e.g. to the vasculature and/or heart, in one-piece, with the first and second structures being connected together, e.g. via the third structure, during delivery. Where the prosthesis includes an artificial aortic valve, the device may be introduced to the aortic valve region in a compact configuration within a delivery sheath inserted via the femoral artery (“transfemoral access”) or inserted through the chest wall and then via the apex of the heart (“transapical access”), although a number of other delivery approaches may be possible. When transfemoral access is undertaken, the delivery sheath may be pre-curved to facilitate passage around the aortic arch and through the native aortic valve.

Transfemoral access with known percutaneous valve devices can be complicated by device misalignment to the aortic valve, associated with the innate angulation of the aortic valve central channel to the aortic vessel walls. This can result in poor function of the valve, or leakage around it. By eccentrically aligning structures of the device itself, a more favourable alignment of the overall device to the valve channel may be achieved.

The prosthesis may be ‘pre-loaded’ into a long sheath, e.g. over a guidewire. The sheath may be curved at its tip in approximate conformity with the profile of the prosthesis when having a preformed eccentrically aligned configuration. This curvature may facilitate passage of the sheath around the curved arch of the aorta, and help align the prosthesis prior to deployment.

Deployment of the prosthesis may be performed under condition of lowered blood pressure (e.g. induced by “over-pacing” or by drug-induced, temporary cardiac asystole). To deploy, an outer sheath may be retracted while a “pusher” catheter holds e.g. the second, e.g. balloon-expandable, structure in position. This second structure may then be expanded by a balloon, and then the sheath may be retracted further to expose and release the first structure. The first structure may be a self-expanding stent, which expands automatically when released from the sheath.

Alternatively, and in accordance with the twelfth and thirteenth aspects discussed below, the prosthesis may be configured to be delivered to the vasculature and/or heart in two or more separate pieces. For example, in one embodiment, connectors of the third structure may be connected to one of the first and second structures during delivery, and only connected to the other of the first and second structures at the deployment location. For example, the connectors may be connected to the second structure during delivery but not to the first structure during delivery, and the first structure may be delivered only after the second structure has been expanded to engage the vasculature and/or heart, whereupon the first structure may engage the connectors, e.g. upon partial expansion of the first structure. By varying a connection region between the connectors and the first and/or second structures at this stage, another approach to adjusting the lengths of the connectors between the first and/or second structures may be provided. Upon engaging the connectors, independent movement of the first and second structures may be possible, e.g. as described with respect to preceding aspects. However, the first structure may be expanded or further expanded to engage the vasculature and/or heart in a manner that may lock the connectors and thus the relative positioning and orientation of the first and second structures.

Alternatively, the first structure may comprise first and second expandable parts, e.g. first and second expandable stents. A first of the parts may be expanded to engage the vasculature and/or heart and a second of the parts may be expanded to press the connectors against the first part, such that at least part of the connectors, e.g. distal ends of the connectors, are sandwiched between the first and second parts of the first structure, fixing the position of the connectors and thus the second structure.

According to a twelfth aspect, there is provided a prosthesis for deployment in the vasculature and/or heart of a body, comprising:

a first expandable structure adapted to engage with an internal surface of the vasculature and/or heart,

a second expandable structure adapted to engage with an internal surface of the vasculature or heart, and

a third structure which is connected to one of the first and second structures and which is connectable to the other of those first and second structures after at least one of those first and second structures has been engaged with an internal surface of the vasculature and/or heart.

According to a thirteenth aspect, there is provided a process for the placement of a prosthesis into the heart and/or vasculature of a body comprising the steps of:

engaging a first structure with an internal surface of the heart or vasculature of a patient;

engaging a second structure with another internal surface of the heart or vasculature of that patient;

and connecting the first and second structures together by a third structure after the first and/or the second structure has been engaged with an internal surface of heart or vasculature of the patient.

The first and second structures may both be configured to engage with the internal surface before the third structure is connected to the other of the first and second structures. Alternatively, the third structure may engage the other of the first and second structures before engaging with the internal surface. Engagement with the respective internal surface may be achieved upon expansion of the first and second structures. The third structure may be connected to, or formed integrally with, either one of the first or the second structure before that first or second structure is engaged with the internal surface.

In a fourteenth aspect there is provided an endoluminal prosthesis for deployment in a body, comprising:

a first expandable structure comprising an outer surface and an inner surface, said inner surface defining a lumen therethrough and at least a region of said outer surface defining a non-cylindrical shape of the first expandable structure when deployed such as to substantially adopt a shape of a surrounding blood vessel and/or part of the heart,

a second expandable structure; and

a third structure connected between the first and second structures,

the third structure having an adjustable shape such as to permit displacement of one of the first and second structures relative to the body independently of displacement of the other of the first and second structures relative to the body.

The prosthesis of the fourteenth aspect may be configured to support a valve member configured to replace or repair a heart valve such as the aortic valve. Typically, the first expandable member supports a valve member.

The second expandable structure, when deployed, is configured to locate at least partially in the ascending aorta whereas the first expandable member is configured to support the valve member and thus is configured to locate at least partially across the annulus of the native aortic valve. The third structure is configured to connect the first and second expandable members and when implanted may locate at, or adjacent to, the sinotubular junction of the aorta where the right and left coronary arteries meet the aorta. As such, the third structure is configured to allow maximum flow of blood through to the coronary arteries.

In one embodiment, a cross section of at least a region of the first expandable structure of the fourteenth aspect normal to a main axis may be circular. In one embodiment, the outer surface of the expandable structure defines a shape having such a circular cross section. Alternatively, the inner surface defines a circular cross section of the expandable structure. In a still further embodiment, the inner surface of the first expandable structure has a circular cross section but the outer surface of said expandable structure has a non-circular cross section; or vice versa. One embodiment of such a generally non-cylindrical structure having at least a region with a circular cross section comprises a frusto-conically shaped expandable member. At least a portion of the first expandable member is frusto-conical although it is also envisaged that such an expandable member may also comprise a portion which is not frusto-conical. The frusto-conical portion of this embodiment may be positioned in the mid-sinusal level of the aorta above the valve ring. The wall of the aorta at this region bulges outwardly from the ventriculo-arterial junction and thus the frusto-conical shape of the first expandable structure is configured to optimally align with the wall of the vessel at this region. The frusto-conical portion of the first expandable structure may extend from a superior end to an inferior end wherein a portion of the first expandable structure adjacent said inferior end is non-frusto-conical. Said non-frusto-conical portion of the expandable structure is configured to sit beneath the frusto-conical portion when in situ and typically sits across the native annulus.

The frusto-conical portion of the first expandable structure may exert a weaker radial force when in situ than, for example, the second expandable member. Still further, the frusto-conical portion of the expandable structure may exert a weaker radial force when in situ than the non-frusto-conical portion.

The frusto-conical portion of the first expandable member may be made from a relatively thinner material. In one embodiment wherein the frusto-conical member comprises a stent, the stent may be made from a suitably flexible and optionally shape memory material. The stent may be made up of a series of cells defined by wireforms of the material. The material of the stent may be relatively thin when compared to other regions of the stent or the second expanding structure such as to allow for the desired weaker radial force when in situ. Alternatively, the desired radial force may be achieved by the design of the cell pattern of the stent eg by increasing the cell size of the stent wall, the stent is relatively weaker and thus exerts less radial force when in situ.

A relatively weaker radial structure and the frusto-conical shape of this embodiment of the first expandable structure has the advantage that the pressure exerted upon the frusto-conical portion when in situ, from the bloodflow therethrough, forces the frusto-conical portion against the wall of the aorta, both during systole and diastole. Such a structure, therefore significantly reduces the likelihood of leakage of the blood around the edges of the prosthesis.

In another embodiment said first expandable member may be connected to a fourth structure. Said fourth structure may comprise a radially expandable stent for positioning generally posterior to the ventriculo-aortic junction. The expandable stent of this embodiment may be configured or made of a suitable material such that it exerts a relatively lesser radial force than, for example, the non-frusto-conical portion of the above embodiment or a portion of the structure of the above aspects which sits across the native aortic valve. While a relatively greater radial force may be required to force against an often calcified region of the aortic valve, a stent or other structure exerting the same forced beneath the native aortic valve annulus could lead to damage of the delicate surrounding tissue such as the region comprising the Bundle of His. Damage to such regions of the wall tissue may cause damage to conduction fibres and may disrupt the natural pacing of the heart, requiring the implantation of an artificial pacemaker in severe cases.

In this embodiment said fourth structure may include a radial force dampener member. In one embodiment said radial force dampener may comprise a cuff member or a balloon member. Such a radial force dampener further protects the delicate tissue of the heart which is critical for natural pacing.

The fourth structure may be connected to the first expandable structure positioned across the aortic valve region by stabilising connectors. Said stabilising connectors may be fixed, or alternatively, they may be adjustable.

Typically, when the prosthesis is for use in replacing a native aortic valve, the shape defined by the outer surface of the first expandable structure when viewed in cross section may comprise a substantially triangular shape. Alternatively the outer surface of the first expandable structure comprises an oval, elliptical or a polygon shape when the structure is viewed in cross section.

It is preferred that any cross-sectional shape of the first expandable structure as defined by the outer surface is devoid of a sharp corner to avoid damage to the surrounding tissue when positioned in a vessel or in the heart.

Typically when positioned within the region of the heart and aorta to support an artificial aortic valve, the outer surface of the first expandable member is triangular shape in cross section with a base and two extending edges meeting at an apex. The junction regions between respective ends of the base and the extending edges and the junction region of the two extending edges at the apex are rounded to avoid any edges or steps in the overall shape of the expandable structure.

The first expandable structure may comprise a stent. For example, the structure may comprise a balloon expandable and/or self-expanding stent.

In a fifteenth aspect, there is provided a valve prosthesis for deployment in a body, said valve prosthesis having a superior end region and a posterior end region and comprising:

a first expandable structure including an expandable main body and a valve member;

a second expandable tethering member,

said first expandable structure and second tethering structure relatively connected by a third structure, and

a fourth structure extending posteriorly from said first expandable structure;

said valve prosthesis configured to be radially expandable from an initial compact state to an expanded state and, when the prosthesis is in its expanded state, each structure has a radial strength, and wherein said fourth structure has relatively less radial strength than at least part of said first structure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments are now described with reference to the accompanying drawings, in which:

FIG. 1 shows a side view of a prosthesis according to one embodiment;

FIG. 2 shows a side view of a prosthesis according to another embodiment;

FIGS. 3a to 3e show schematic views depicting relative movement of the first and second structures of a prosthesis according to another embodiment;

FIGS. 4a and 4b show schematic views of a prosthesis according to another embodiment;

FIGS. 5a and 5b show schematic views of a prosthesis according to another embodiment;

FIGS. 6a and 6b show a connector of a prosthesis according to another embodiment;

FIGS. 7a to 7c show three prosthesis according to three further embodiments, respectively, and FIGS. 7d to 7h illustrate methods of deploying a prosthesis according to FIGS. 7a to 7c;

FIG. 8a shows a prosthesis according to another embodiment; and FIGS. 8b to 8n illustrate methods of deploying the prosthesis according to FIG. 8a;

FIGS. 9a and 9b show a prosthesis according to another embodiment;

FIG. 10 depicts part of the anatomy of a subject around the aortic valve region;

FIG. 11 depicts in cross section a region of the wall at the aortic valve region;

FIG. 12 is an ultrasound image of the aortic valve of a subject;

FIG. 13 is a schematic view of a prosthesis of a further aspect of the invention;

FIG. 14 depict a longitudinally cross sectional view of the prosthesis of FIG. 13; and

FIG. 15 is a cross sectional view through I-I of FIG. 14.

### DETAILED DESCRIPTION OF EMBODIMENTS

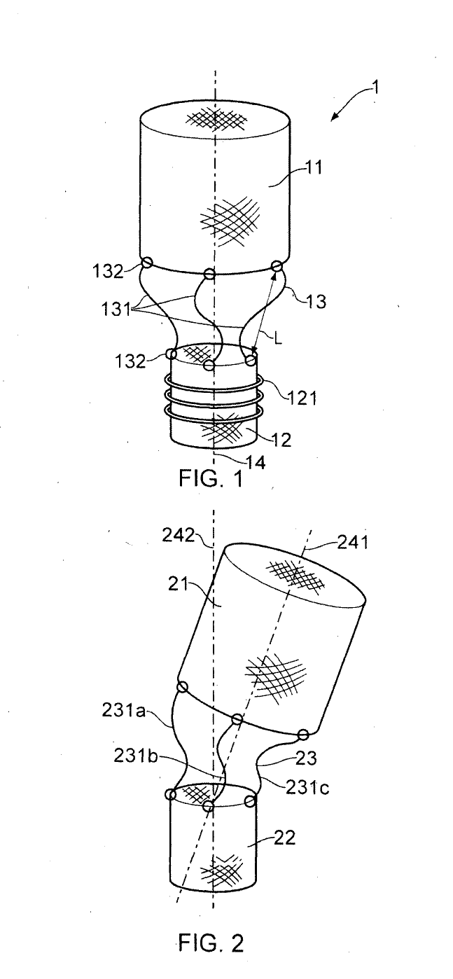

A side view of an endoluminal prosthesis 1 according to an embodiment of the present disclosure is shown in FIG. 1. The prosthesis 1 includes an expandable stabilisation stent 11, providing a first structure of the prosthesis, an expandable valve stent 12, providing a second structure of the prosthesis, the stabilisation and valve stents 11, 12 being space apart, and an expandable middle structure 13, providing a third structure of the prosthesis, the middle structure 13 connecting together the stabilisation and valve stents 11, 12.

In this embodiment, the stabilisation and valve stents 11, 12 are each expandable between compact and expanded states, and are substantially cylindrical, and concentric about a main prosthesis axis 14, in their natural, expanded states. However, the stabilisation stent 11 has a greater diameter than the valve stent 12, consistent with its intended deployment location in the ascending aorta, which normally has a greater diameter than the aortic channel below the sinotubular junction, where the valve stent 12 is to be deployed.

The valve stent 12 is configured in this embodiment to support an artificial heart valve and designed to locate across a damaged (native) aortic valve, maintaining the aortic valve open and ensuring that artificial heart valve can function fully as a replacement valve. Nonetheless, in alternative embodiments, the valve stent 12 may be configured to support an artificial heart valve or alternative device designed to supplement and/or repair the native aortic valve. The valve stent 12 includes a plurality of sealing rings 121 on an outer surface thereof, configured to assist in forming a seal between the stent 12 and surrounding blood vessel walls. The stabilisation stent 11 is configured to anchor the prosthesis 1 in position with respect to the ascending aorta.

The middle structure 13 has an adjustable shape such as to permit not only relative movement of the stabilisation and valve stents 11, 12, but independent movement of the stents 11, 12 relative to the vessels in which they are deployed. Accordingly, the stabilisation stent 11 may be oriented and positioned in the most appropriate position in the aorta without causing a change of the position or orientation of the valve stent 12, and likewise the valve stent 12 may be oriented and positioned in the most appropriate position in the aortic channel without causing a change of the position or orientation of the stabilisation stent 11. This independent moveability of the stents 11, 12 may be particularly useful to the surgeon when fine tuning the positioning of the prosthesis 1 during or after expansion of the various structures 11, 12, and 13.

In the embodiment shown in FIG. 1, the shape of the middle structure 13 is generally defined by connectors 131 that extend between the stabilisation and valve stents 11, 12. The connectors 131 are each formed of flexible material and have a curved, S-shape. Each of the ends of the connectors 131 is connected to the stabilisation and valve stents 11, 12 via respective connection rings 132. The rings 132 provide for articulation between the connectors 131 and the stents 11, 12, allowing the connectors 131 to rotate relative to the stents 11, 12.

By forming the connectors 131 of curved, flexible material, the lengths of the connectors between the stabilisation and valve stents 11, 12 can be varied. In particular, the length of each connector 131 between the stents 11, 12 can be increased by decreasing the degree of curvature of the connector 131, and thus straightening out the connector 131, and the length can be decreased by increasing the degree of curvature of the connector 131. Generally, references herein to the length of connectors 131 between the stabilisation and valve stents describe a straight-line distance between the connection points of the connectors and the stabilisation and valve stents respectively, as indicated by line L in FIG. 1, for example. In this embodiment, the connectors 131 are not interconnected, and the lengths of the connectors 131 between the stabilisation and valve stents 11, 12 can be varied relative to each other, in addition to being variable in unison.

A side view of a prosthesis 2 according to another embodiment is shown in FIG. 2. The prosthesis 2 includes an expandable stabilisation stent 21, providing a first structure of the prosthesis, an expandable valve stent 22, providing a second structure of the prosthesis, the stabilisation and valve stents 21, 22 being space apart, and an expandable middle structure 23, providing a third structure of the prosthesis, the middle structure 23 connecting together the stabilisation and valve stents 21, 22. Generally, the structures 21, 22, 23 of FIG. 2 have similar form and function to the structures 11, 12, 13 described above with respect to FIG. 1. However, the shape of the middle structure 23 differs from the shape of the middle structure 13 in a manner that causes a naturally eccentric (angulated) alignment between the stabilisation and valve stents 21, 22. In particular, the dimensions of the connectors 231a-c forming the middle structure are different from one another, such that, at least in a natural, fully expanded state, the cylindrical stabilisation stent 21 (shown concentric with a first axis 241 in FIG. 2) is not concentric with the cylindrical valve stent 22 (shown concentric with a second axis 242 in FIG. 2). Although the lengths of the connectors 231a-c between the stents 21, 22 are adjustable as described above with respect to FIG. 1, the adjustability is from a starting point where the stabilisation and valve stents 21, 22 are eccentrically aligned. The eccentric alignment may more naturally follow the anatomy of the aortic root and valve, where an eccentric alignment between the ascending aorta and the aortic channel below the sinotubular junction is commonly found.

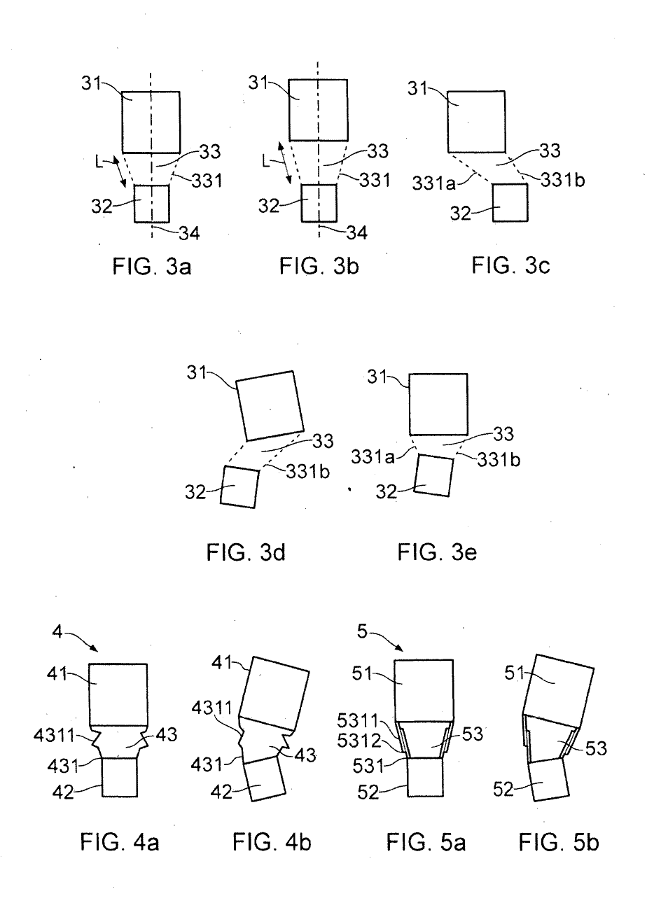

FIGS. 3a to 3e show schematic examples of how the lengths L of connectors 331 between first and second structures 31, 32 (e.g. stabilisation and valve stents as described with respect to FIGS. 1 and 2), and thus the shape of a middle structure 33, can vary to accommodate changes in the relative orientation and position of first and second structures 31, 32. In FIGS. 3a to 3e, connectors 331 on directly opposite sides of the prosthesis are shown for simplicity only, although a number of additional connectors will normally be provided around a circumference of the prosthesis.

In FIG. 3a, the structures 31, 32 are aligned concentrically about a main axis 34, with the lengths of the opposing connectors 331 being substantially equal. In FIG. 3b, the lengths of the connectors 331 have been adjusted in unison (in equal measure), moving the structures 31, 32 apart from the configuration shown in FIG. 3a, while maintaining the concentric alignment. In FIG. 3c, the connectors 331a on one side of the prosthesis have increased in length in comparison to the connectors 331b on an opposite side, in a manner that has allowed a translational movement of the structures 31, 32 from the configuration shown in FIG. 3a. In FIG. 3d, the connectors 331a on one side have decreased in length in comparison to the connectors 331b on the opposite side, in a manner that has allowed both translational and rotational movement of the structures 31, 32 from the configuration shown in FIG. 3a. In FIG. 3e, the connectors 331a on one side have decreased in length in comparison to the connectors 331b on the opposite side, yet all of the connectors 331a, 331b have decreased in length in a manner that has allowed both rotational movement of the structures 31, 32 and movement of the structures 31, 32 towards each other from the configuration shown in FIG. 3a.

FIGS. 4a and 4b, and FIGS. 5a and 5b, show examples of alternative types of length-variable connectors that may be used in the middle structure of embodiments of the present disclosure.

In more detail, referring to FIGS. 4a and 4b, a prosthesis 4 according to one embodiment is shown. The prosthesis 4 includes first and second structures 41, 42 (e.g. stabilisation stent and valve stents), and a middle structure 43 connected between the first and second structures 41, 42. The configuration of the prosthesis 4 of this embodiment can be similar to the prosthesis 1 described above with respect to FIG. 1; however, the connectors 431 each have a zigzag region 4311 instead of a curved, S-shape. As can seen by comparing FIGS. 4a and 4b, by straightening out or compressing the zigzag region 4311, the lengths of the connectors 431 between the first and second structures 41, 42 can be increased or decreased, respectively, making relative movement of the first and second structures 41, 42 possible.

Referring to FIGS. 5a and 5b, a prosthesis 5 according to yet another embodiment is shown. The prosthesis 5 includes first and second structures 51, 52 (e.g. stabilisation stent and valve stents), and a middle structure 53 connected between the first and second structures 51, 52. The configuration of the prosthesis 5 of this embodiment can be similar to the prosthesis 1 described above with respect to FIG. 1; however, the connectors 531 are each formed of first and second relatively slidable portions 5311, 5312. As can seen by comparing FIGS. 5a and 5b, by sliding the first and second portions 5311, 5312 relative to each other, the lengths of the connectors 531 between the first and second structures 51, 52 can be varied, making relative movement of the first and second structures 51, 52 possible.

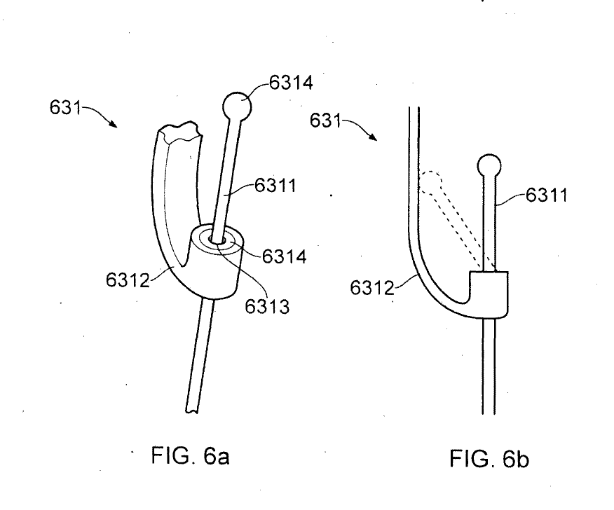

In another embodiment, with reference to FIGS. 6a and 6b, first and second slidable portions of a connector 631 are provided by first and second arms 6311, 6312, the first arm 6311 being substantially straight and the second arm 6312 having a bent region with an orifice 6313 lined by a rubber ring 6314 at one end through which the first arm 6311 is slidably mounted to the second arm 6312. The first arm 6311 is connected to one of first and second structures and the second arm 6312 is connected to the other of the first and second structures. By relatively sliding the first and second arms 6311, 6312, the length of the connector 631 can be varied generally as described with respect to FIGS. 5a and 5b. The first arm 6311 has a stop 6314 at one end to prevent the first arm 6311 being passed entirely through the orifice 6313, preventing disengagement of the first and second arms 6311, 6312 and providing a limit to the length variation of the connector 631.

When a desired relative positioning of the first and second structures has been achieved, the length of the connector 631 can be substantially fixed by deforming the first arm 6311 in a manner that prevents further sliding of the first arm 6311 relative to the second arm 6312. To this end, the first arm 6311 can be formed of an annealed material that can be bent e.g. upon exertion of force by a balloon, radially outwardly as shown in FIG. 6b.

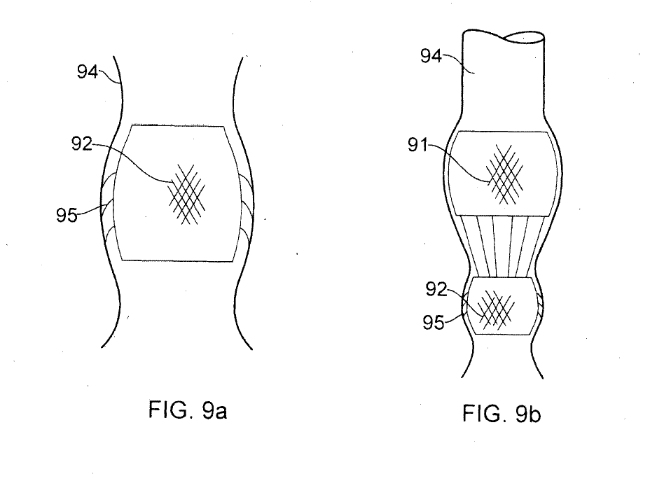

Although the stabilisation and valve stents of the preceding embodiments are generally shown as cylindrical in nature, they make take other forms. For example, with reference to FIGS. 9a and 9b, the outer sides of stabilisation and/or valve stents 91, 92 may be convex, such that the prosthesis has an ability to self align within a vessel 94 and/or is more easily alignable with the vessel 94 by a surgeon. This may provide the stents 91, 92 with a barrel-shaped configuration and may ensure that the stents are stronger than stents with straight sides. One or more of the stents, e.g. the valve stent 92, can be fitted with fine sealing rings 95 made from silicone, rubber or soft plastic, for example, to assist in sealing against the walls of the vessel 94. Between the sealing rings 95, a polyester mesh or similar material may be provided, which will coagulate with the presence of blood and prevent backflow of blood, assisting in anchoring of the valve stent.

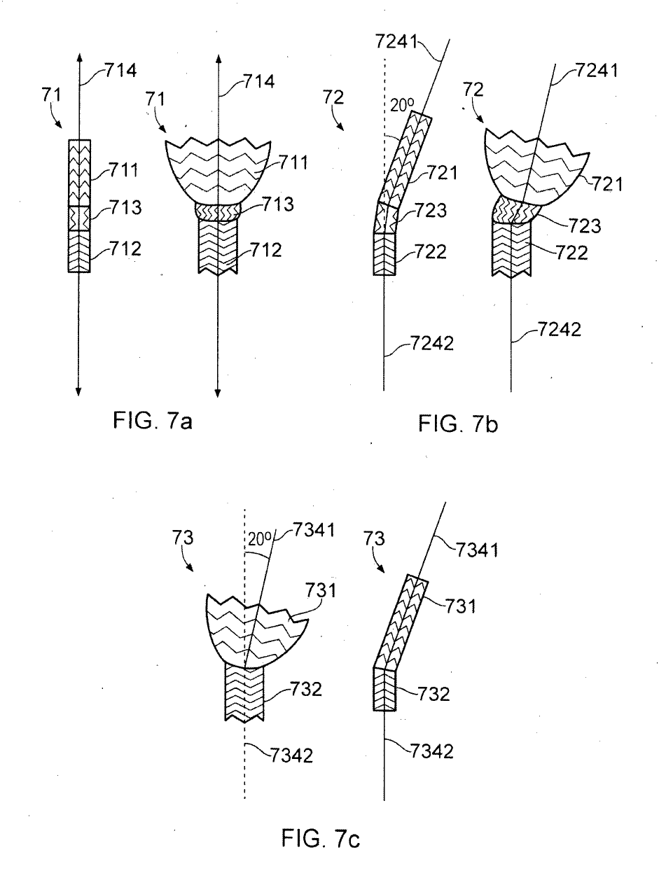

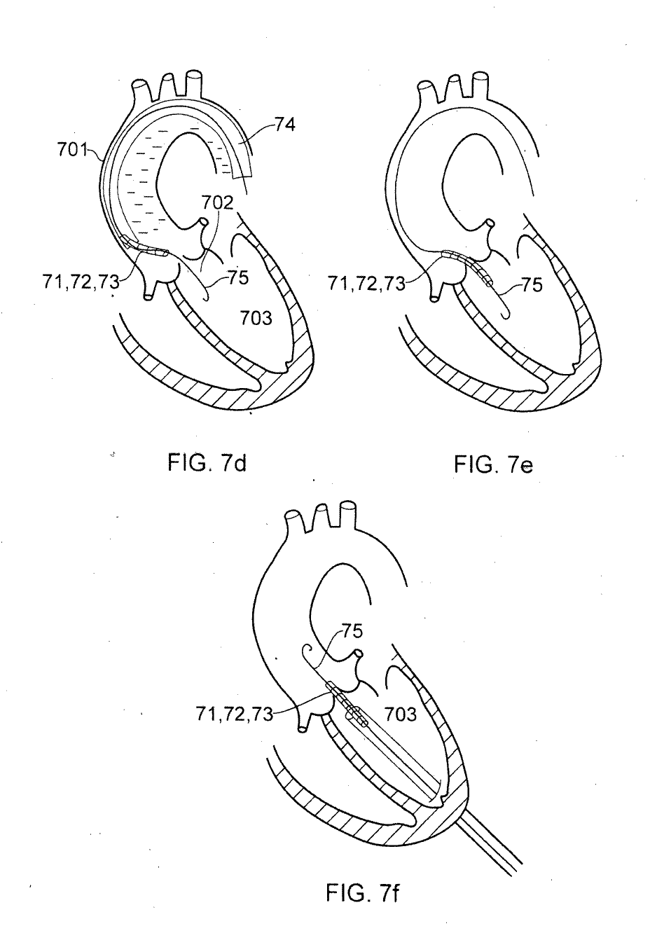

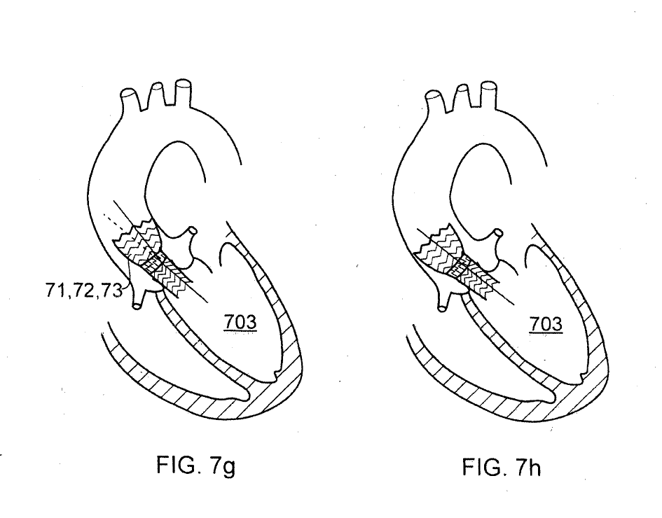

Three different prosthesis 71, 72, 73 according to three further embodiments are shown in FIGS. 7a to 7c, respectively. The prosthesis 71, which is shown both in a compact and expanded state FIG. 7a, includes a self-expandable stabilisation stent 711, providing a first structure of the prosthesis 71, a balloon expandable valve stent 712, providing a second structure of the prosthesis 71 that supports an artificial heart valve, and an expandable middle structure 713, providing a third structure of the prosthesis 71, the middle structure 713 being configured to connect together the stabilisation and valve stents 711, 712. In this embodiment, the stabilisation and valve stents 711, 712 are concentric about a single main axis 714 in their compact and natural, fully expanded, states, but the middle structure 713 is flexible and adaptable, generally as described with respect to preceding embodiments, such that the stabilisation and valve stents 711, 712 can be eccentrically aligned during delivery and/or deployment, e.g. so that the stabilisation and valve stents 711, 712 are concentric about different, relatively angled, axes.

The prosthesis 72 shown in FIG. 7b is substantially identical to the prosthesis 71, except that it includes stabilisation and valve stents 721, 722, connected together via a middle structure 713, which are concentric about different, relatively angled, axes 7241, 7242, both in their compact and natural, fully expanded states. Essentially, the stabilisation and valve stents 721, 722 can be considered pre-angulated, e.g. by about 10 or 20 degrees in this embodiment. The structures 721, 722, 723 have a slight flexibility, which allows minor angle variations, but essentially prevents loss of the angulation between the stents 721, 722. Generally, in FIG. 7b, the stabilisation stent 721 and the middle structure 723 are shown aligned with each other along the axis 7241, and angulated with respect to the valve stent 722. However, in alternative embodiments, the middle structure 723 may be aligned with the valve stent 722, and angulated with respect to the stabilisation stent 721.

The prosthesis 73 shown in FIG. 7c is substantially identical to the prosthesis 72, except that it includes stabilisation and valve stents 731, 732 that are directly connected to each other, that is without any interconnecting middle structure. Again, the stabilisation and valve stents 731, 732 are concentric about different, relatively angled, axes 7341, 7342, both in their compact and natural, fully expanded states, and thus the stabilisation and valve stents 731, 732 can be considered pre-angulated, e.g. by about 10 or 20 degrees in this embodiment. The structures 731, 732, also have a slight flexibility, which allows minor angle variations, but essentially prevents loss of the angulation.

Delivery and deployment methods for the prostheses 71, 72, 73 illustrated in FIGS. 7a to 7c are now described with reference to FIGS. 7d to 7h.

Referring to FIG. 7d, in one embodiment delivery of the prosthesis 71, 72, 73 to the aortic root is carried out via the femoral artery (“transfemoral access”). The prosthesis 71, 72, 73 is ‘pre-loaded’ into an outer sheath 74, over a transfemoral guidewire 75 inserted through the ascending aorta 701, through the aortic valve 702 and into the left ventricle 703. The sheath 74 in this embodiment is curved at its distal end in approximate conformity with the profile of the angulated prosthesis 71, 72, 73. This curvature facilitates passage of the sheath around the curved arch of the aorta, and helps align the prosthesis 71, 72, 73 prior to deployment with the aortic valve channel. The use of the curved sheath prevents the prosthesis 71, 72, 73 becoming misaligned with the aortic valve channel as otherwise represented in FIG. 7e.

Referring to FIG. 7f, in another embodiment the prosthesis 71, 72, 73 is pre-loaded into a trans-apical access sheath that is inserted through the chest wall and then via the apex of the heart (“transapical access”).

Deployment is performed under condition of lowered blood pressure (induced by “over-pacing” or by drug-induced, temporary cardiac asystole). For deployment, the outer sheath 74 is retracted while a pusher catheter holds the balloon-expandable valve stent 712, 722, 732 in position. This stent 712, 722, 732 is then expanded by a balloon, and then the sheath 74 is retracted further to expose and release the self-expanding stabilisation stent 711, 721, 731. The valve stent 712, 722, 732 includes an artificial valve structure attached to an inner surface thereof either by suturing, glues or other physical and/or chemical means.

The prosthesis 71, 72, 73 having an angulated configuration is shown in its deployment position in FIG. 7g. In comparison to FIG. 7h, which shows a non-angulated prosthesis in a similar deployment position, the prosthesis 71, 72, 73 aligns more favourably with the anatomy at the aortic root and valve.

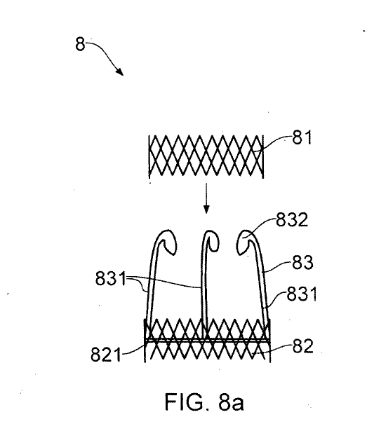

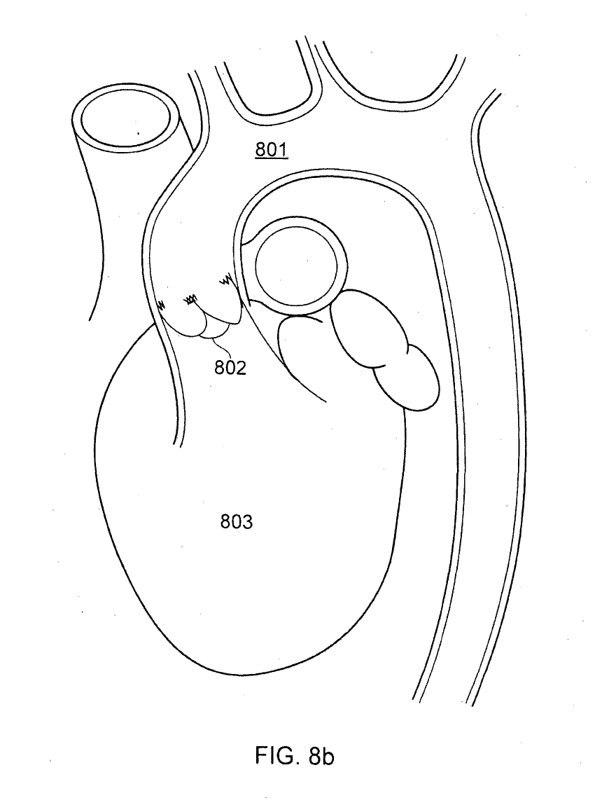

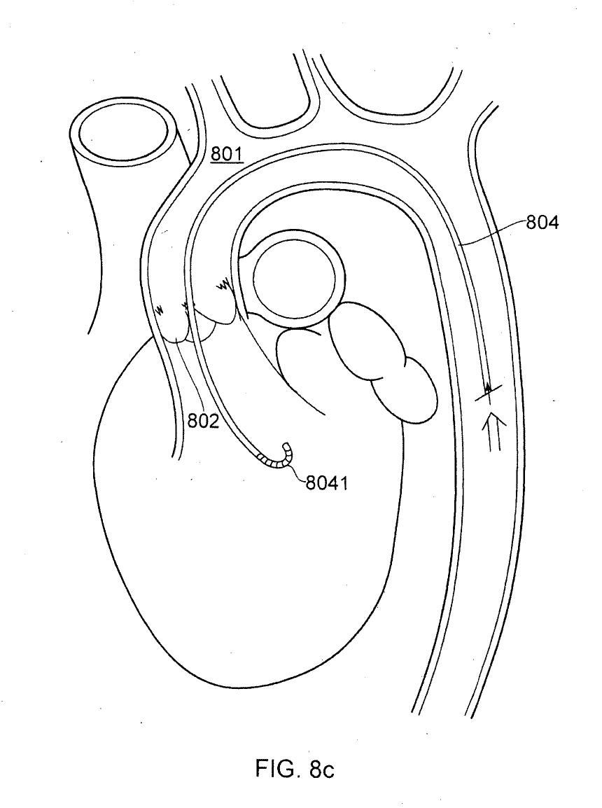

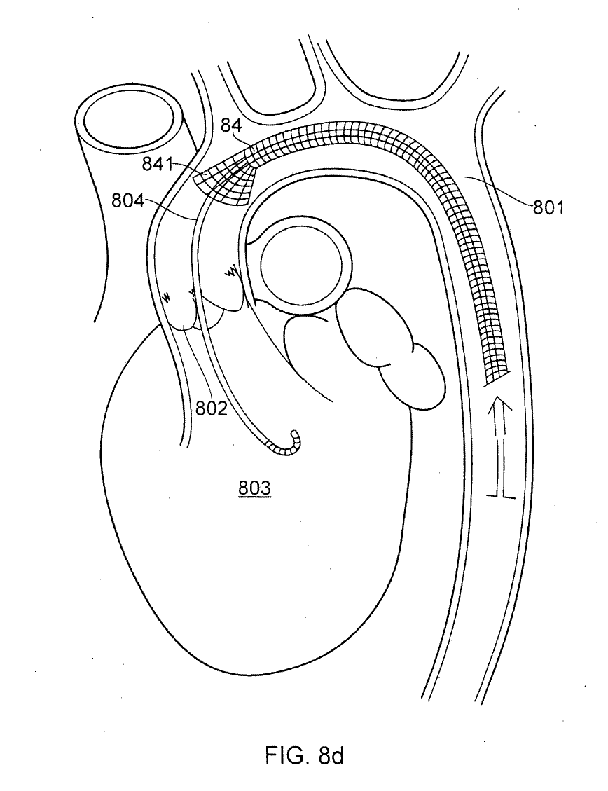

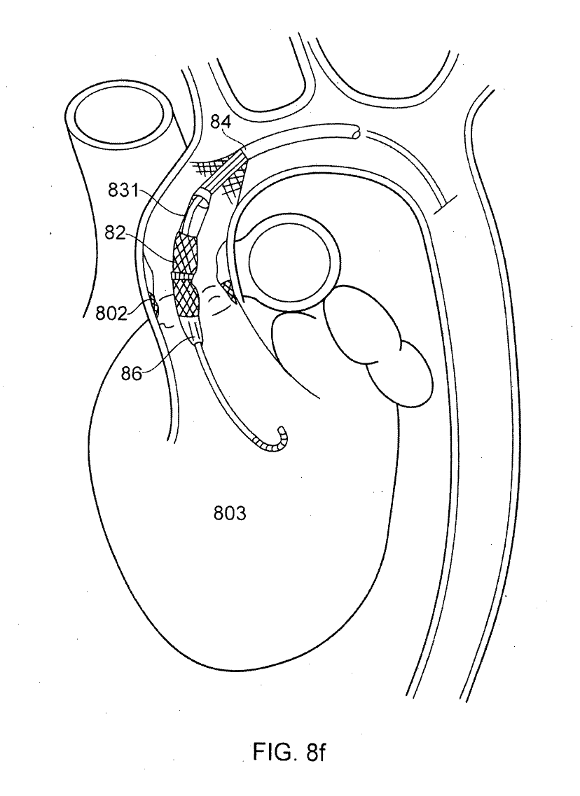

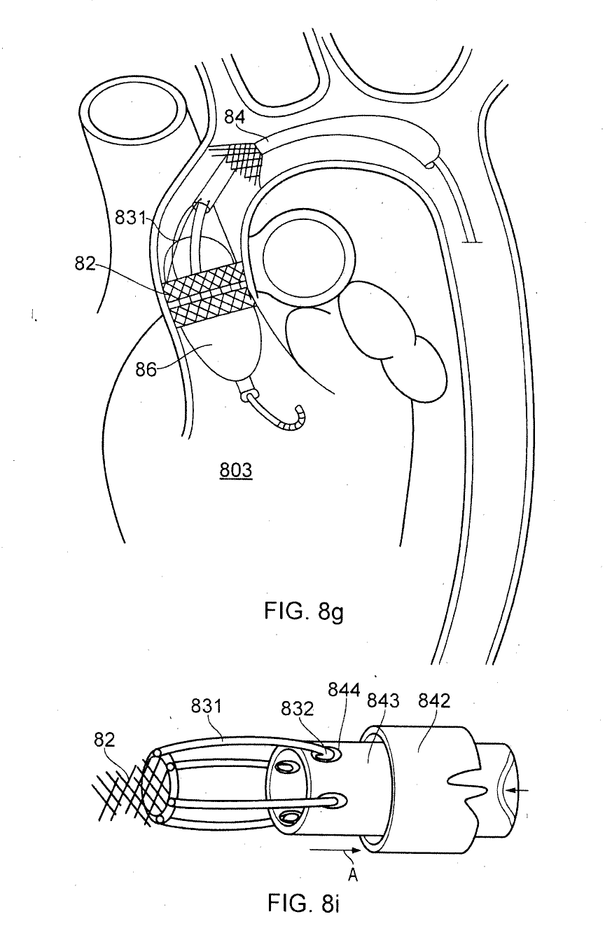

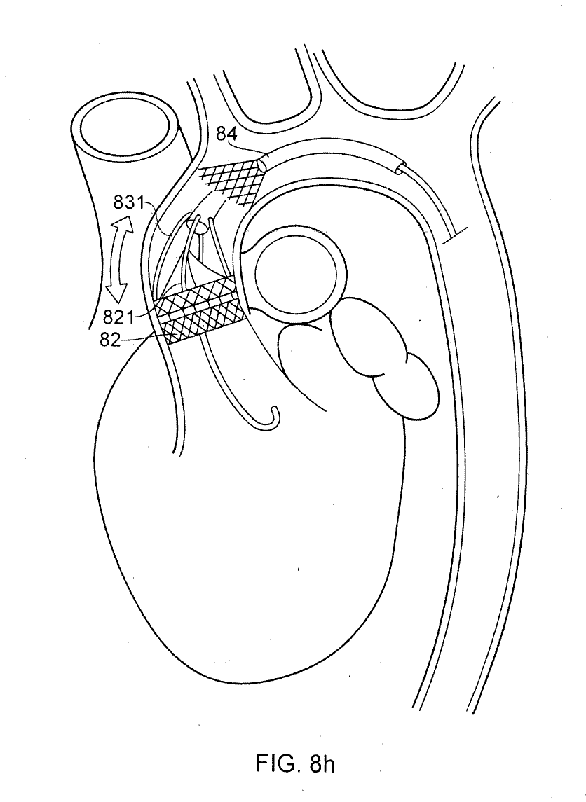

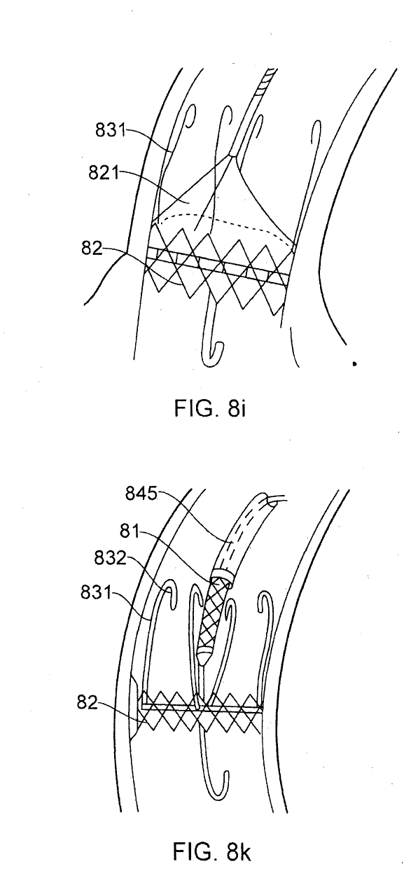

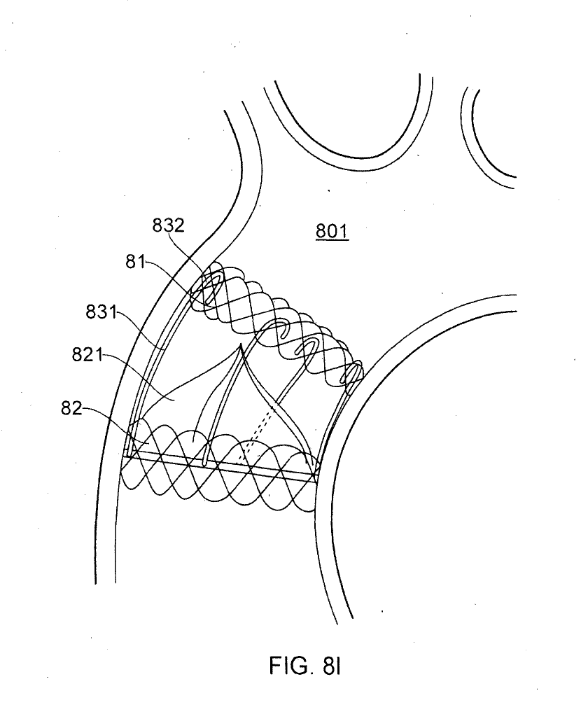

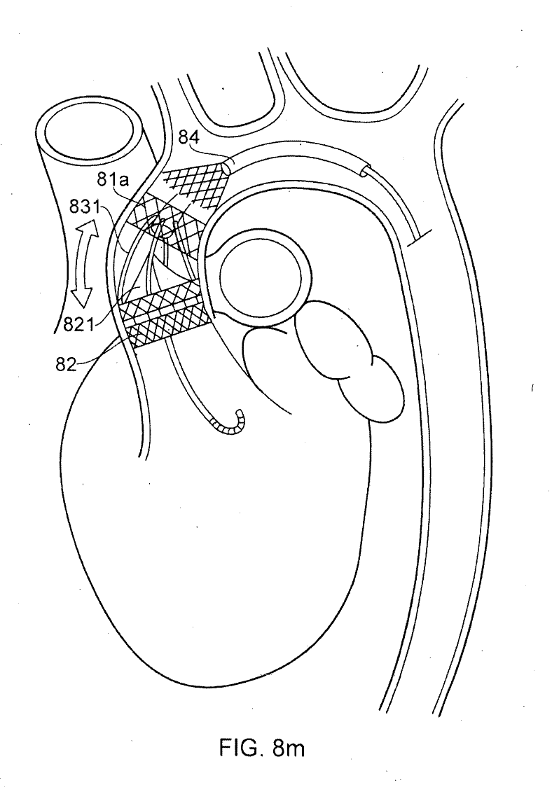

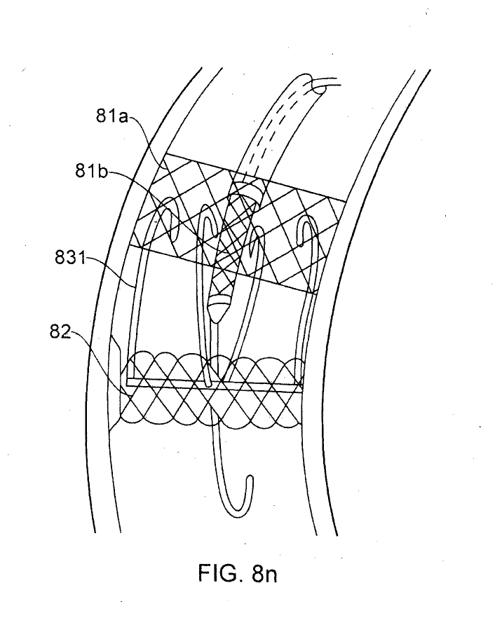

A side view of a prosthesis 8 according to another embodiment is shown in FIG. 8a. The prosthesis 8 includes an expandable stabilisation stent 81, providing a first structure of the prosthesis 8, an expandable valve stent 82, providing a second structure of the prosthesis that supports an artificial heart valve 821, and an expandable middle structure 83, providing a third structure of the prosthesis, the middle structure 83 being configured to connect together the stabilisation and valve stents 81, 82. In this embodiment, the stabilisation and valve stents 81, 82 are separable such that the prosthesis 8 is configured to be delivered to a vascular site in separate parts. In particular, during delivery, the third structure 83, which comprises a plurality of connectors 831 having hooked shaped paddles 832 at their distal ends, is fixed to the valve stent 82 but is separated from the stabilisation stent 81. The arrangement is such that the valve stent 82 can be positioned and oriented as appropriate independently of the stabilisation stent 81, prior to full or partial delivery of the stabilisation stent 82, which is subsequently connectable to the third structure by engaging the paddles 832 so as to stabilise the valve stent 82. Although the third structure 83 is fixed to the valve stent and separated from the stabilisation stent during delivery in this embodiment, in alternative embodiments the third structure may be fixed to the stabilisation stent 81 and separated from the valve stent during delivery. An exemplary delivery method for the prosthesis of FIG. 8a is now described.

Referring to FIG. 8b, the prosthesis 8 is configured to be repair or replace a stenotic native aortic valve 802 located at the opening between the left ventricle 803 and the ascending aorta 801.

Referring to FIG. 8c, a guide wire 804 is inserted into the ascending aorta 801 and extended through the aortic valve 802, such that the distal end 8041 of the guidewire 804 locates in the left ventricle 803. The guide wire 804 used in this embodiment is a j-wire having a j-shaped distal end 8041.

Referring to FIG. 8d, a delivery device 84 is passed over the guidewire 804 and advanced towards the aortic valve 802. At the distal end of the delivery device 84, a conical shaped filter. 841 is provided that extends laterally across the ascending aorta 801, preventing e.g. calcified matter passing through the ascending aorta when the aortic valve 802 is opened.

Referring to FIG. 8e, the delivery device includes an outer sheath 842 that extends over the guide wire 804 and which accommodates a valvuloplasty balloon 85 in a deflated state, as it is advanced towards the aortic valve 802. When the distal end of the device reaches the aortic valve 802, the outer sheath 842 is partially withdrawn to reveal the balloon 85, which is subsequently inflated to force the aortic valve 802 into an open position. To ensure that the balloon 85 is appropriately positioned to expand and open the valve 802, the balloon includes position markers 851 at opposite ends. Subsequent to opening of the valve 802, the valvuloplasty balloon 85 is deflated and withdrawn.

Referring to FIGS. 8f to 8h, the valve stent 82 is then advanced through the delivery device 84 into a position where it extends through the open valve 802 in a compact non-expanded state. Manipulation of the valve stent 82 into an appropriate initial position and orientation is achieved through holding, by the delivery device 84, of the distal ends of the connectors 831 that extend from the valve stent 82. The valve stent 82 is expanded and deployed using a low pressure balloon 86, while the distal ends of the connectors 831 continue to be held by the delivery device 84. Since the low pressure balloon 86 causes only a relatively light engagement between the valve stent 82 and the walls of the aorta/the native aortic valve 802 upon expansion, the delivery device 84 can be used to fine tune the positioning and orientation of the valve stent 82 even after expansion of the valve stent 82 and after deflation and removal of the low pressure balloon 86.

Referring to FIG. 8i, the paddles 832 at the distal ends of the connectors 831 are located within respective recesses 844 provide circumferentially about the outer surface of an inner sheath 843 of the delivery device 84. The inner sheath 843 provides a lumen for the low pressure balloon 86. Initially, the outer sheath 842 locates over the paddles 832 to maintain the paddles 832 in the recesses 844 even after the valve stent 82 is expanded using the low pressure balloon 86, allowing the delivery device 84 to hold and manipulate the positioning and orientation of the valve stent 82 as described above. To release the connectors 831 from engagement with the delivery device 84, the outer sheath 842 is translated relative to the inner sheath 843 in the direction indicated by arrow A, causing the paddles 832 to spring out of the recesses 844 due to the potential energy generated previously by the expansion of the valve stent 82. After the paddles 832 exit the recesses 844, the connectors 831 align substantially with the inner surface wall of the aorta, as seen in FIG. 8j.

Subsequently, with reference to FIG. 8k, the self-expandable stabilisation stent 81 is delivered in a sheath 845, where it is held in a compact non-expanded state, to a position where it aligns with the paddles 832 of the connectors 831. The sheath 845 is then withdrawn such as to release the stabilisation stent 81, whereupon the stabilisation stent 81 automatically expands outwardly and presses against, and engages, the hooked-shaped paddles 832 of the connectors 831, as shown in FIG. 8l. The stabilisation stent 81 maintains a relatively strong engagement with walls of the surrounding vessel in comparison to the valve stent 82, serving to anchor the valve stent 82 in position, and maintain patency of the surrounding vessel.

As a variation of the deployment method steps described above with respect to FIGS. 8j to 8l, the stabilisation may be achieved using first and second self-expanding stabilisation stents 81a and 81b. The first stabilisation stent 81a may be expanded prior to release of the connectors 831 from engagement with the delivery device 84, as shown in FIG. 8m. Thus, the first stabilisation stent 81a may function to maintain patency of the aortic wall, while fine tuning of the positioning and orientation of the valve stent 82 using the delivery device 84 remains possible. The connectors 831 may then be released as shown in FIG. 8n, in a manner described with respect to FIG. 8i, whereafter the second stabilisation stent 81b can then be expanded to fix the positions of the connectors, and thus the valve stent 82, by sandwiching the paddles 832 of the connectors 831 between the first and second stabilisation stents 81a, 81b.

In one or more embodiments of the present invention, a prosthesis 100 is provided to provide an optimal fit within the vessels/heart of patients.

The aorta and heart around the aortic valve region is not uniform and thus cylindrically shaped stents may often slip or allow the leaking of blood (regurgitation) back into the ventricle during or just after systole or from the ventricle to the aorta during the diastolic phase.

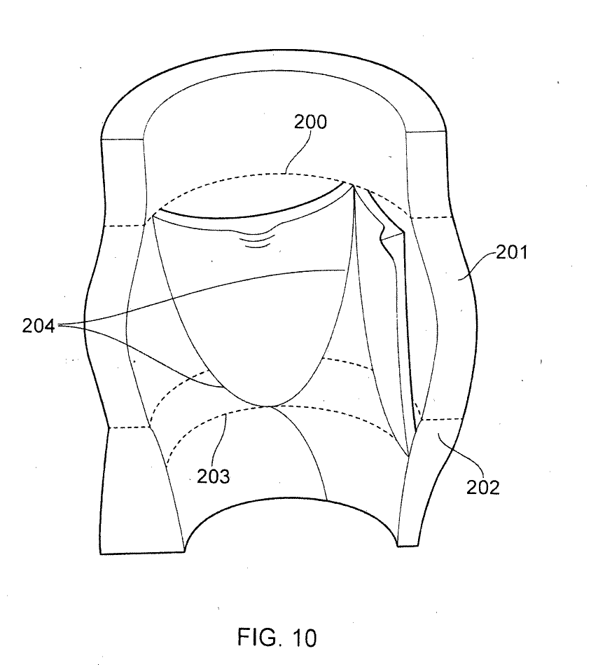

FIG. 10 shows a cross sectional schematic view of the region of the vasculature around the aortic valve including the sinotubular junction of the aorta 200, the mid sinusal level of the aorta 201, the anatomic ventriculo-arterial junction 202 and the virtual basal ring 203. The semilunar attachments 204 are also shown.

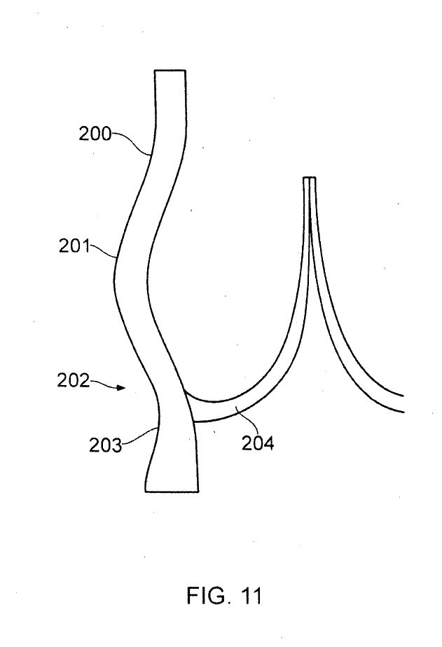

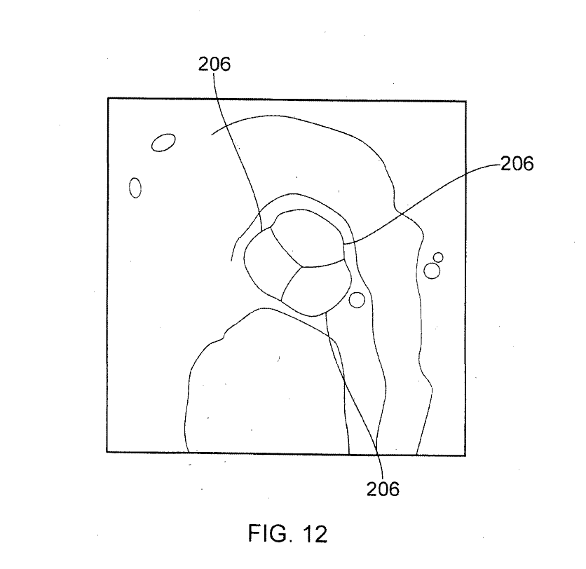

In the exploded cross sectional view of FIG. 11, it can be seen that the mid-sinusal level forms a outward bulge of the vessel wall. As most prostheses sit across this region, it is important to provide the best fit therein. Furthermore, not only is the vessel wall irregular longitudinally, but in cross section across a plane normal to the longitudinal axis of the aorta, the region of the aortic valve is not a perfect circle but rather, the cross section is typically oval or elliptical. The ultrasound image in FIG. 12 provides an image of the aortic valve in cross section and it can be seen that the wall 206 around the valve defines a non-circular shape. Conventional cylindrical stents sitting within this region therefore do not provide an optimal fit. Indeed, with such stents, considerable radial force is applied to force the cylindrical stent into the shape of the surrounding vessel. Such increased radial force can damage the nerve fibres in the tissue and when used in the aortic valve region this could potentially damage the conductive fibres, causing pacing defects, which in the most severe cases require the patient to have an artificial pacemaker fitted.

The aspects and embodiments disclosed herein aim to provide an optimal fit of a prosthesis, while providing a good anchoring to prevent slippage once in situ.

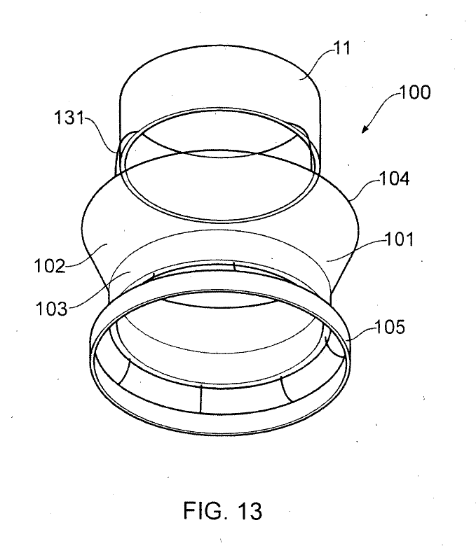

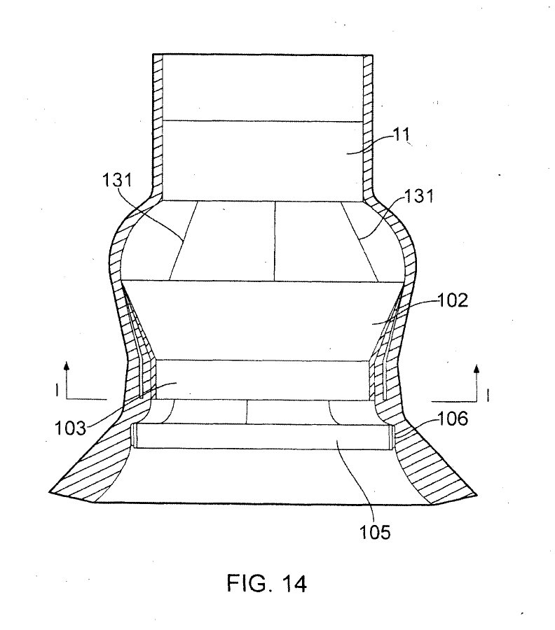

Prosthesis 100 of FIG. 13 is one example of a suitable prosthesis to support an artificial valve. Prosthesis 100 comprises a main body 101 having a first frustoconical region 102 and a second non-frustoconical region 103. While both regions may have a cylindrical cross section, it is preferred that the cross section of the device normal the main axis is non-circular, and including oval or elliptical, or substantially triangular in shape.

Frustoconical region 102 is positioned within the mid-sinusal level of the aorta when the prosthesis 100 is deployed in a patient. Typically, an upper edge 104 may be aligned with the greatest diameter of the aorta at the mid sinusal region 201 and as shown as 204 in FIG. 10. Alternatively, the frustoconical region 102 extends to the sino-tubular junction of the aorta and below the coronary ostia.

Non-frustoconical region 103 is configured to sit inferior relative to the frusto-conical region 102 in situ and may be positioned between the virtual basal ring 203 and the anatomic ventriculo-arterial junction 202.

Main body 101 may be used alone for replacing an aortic valve of a patient. Alternatively, main body 101 may be connected to a stabilising stent as per the above discussion and wherein connectors equivalent to connectors 131 may connect main body 101 to a stabilisation stent equivalent to stabilisation stent 11 discussed above.

The features of the stabilisation stent 11 and the connectors 131 discussed above are herein incorporated in relation to FIGS. 13 and 14.

Prosthesis 100 may comprise a further anchor member 105. Anchor member 105 comprises a stent and optionally at least one cuff or balloon member (not shown) disposed thereon. Anchor member 105 typically sits beneath the virtual basal ring and acts to anchor the main body 101 when deployed in situ. Given the delicate nature of the tissue and the fact that the Bundle of His is located in this region, anchor member 105 may be radially expanded in situ and exert less radial pressure on the surrounding vessel wall than is required for main body 101. Still further, having a cuff or balloon disposed thereon will protect the surrounding delicate tissue still further by dampening the radial force applied thereto. The anchor member 105 may also comprise micro-hook members 106 around all or at least a part of an outer facing surface to optimise attachment to the surrounding vessel wall.

The embodiment depicted in, for example, FIG. 13 has the advantage that anchoring of the device may be easily achieved upon initial deployment of anchor member 105 beneath the native annulus of a patient. In this regard, it is envisaged that the entire assembly prosthesis 100 may be delivered as a unitary member within a single delivery catheter. Typically, the prosthesis 100 is packaged around a balloon (not shown) although the prosthesis 100 may also be self expanding. The prosthesis may be partially self expandable and partially balloon expandable. In this embodiment, the prosthesis 100 is delivered through the aortic arch in a delivery assembly until a distal end of the delivery assembly extends beyond the native annulus of the patient and slightly into the left ventricle. At this time, the catheter sheath surrounding the prosthesis 100 may be partially withdrawn to allow release of anchor member 105. Positioning of anchor member may be relatively straightforward and involves a slight longitudinal retraction/pulling of the deployed anchor member 105 until a resistance is felt. With the knowledge that the anchor member 105 is properly positioned and because the other component structures of prosthesis 100 are of relatively fixed spacing from one another to allow for correct positioning in situ, a surgeon may then simply withdraw the catheter sheath to allow the other components to radially expand in their correct position. A balloon catheter may subsequently be introduced to further expand various parts of the prosthesis 100.

The relative lack of radial strength of the anchor member 105 which is necessary to prevent damage to the heart tissue, is compensated for by the stabilisation stent 11 which also acts to hold the prosthesis in place and prevent slippage.

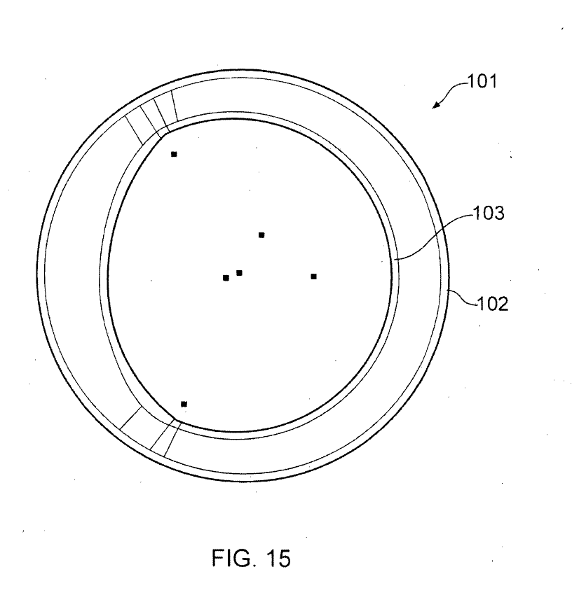

FIG. 15 is a cross sectional view of main body 101 and shows the non-circular cross section of at least the non-frustoconical region 103. While frustoconical region 102 is depicted as having a circular cross section in this figure, it is to be appreciated that it may have the same cross sectional shape as the non-frustoconical region 103 or a different but still non-circular cross section relative to the non-frustoconical region 103

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1-23. (canceled)

24. An endoluminal prosthesis for deployment in a body, comprising:
a first expandable structure,
a second expandable structure; and
a third structure connected between the first and second structures,
wherein the third structure comprises a plurality of connectors connected between the first and second structures, the lengths of the connectors between the first and second structures being adjustable relative to each other.

25. The endoluminal prosthesis of claim 24 configured to be radially expandable from an initial compact state to an expanded state and, when the prosthesis is in its expanded state, each structure has a radial strength, and wherein said structures have variable radial strength relative to one another.

26. The endoluminal prosthesis of claim 24 for deployment in the vasculature and/or heart of a subject.

27. The endoluminal prosthesis of claim 24 wherein said second structure comprises a prosthetic device.

28. The endoluminal prosthesis of claim 27 wherein said prosthetic device is an artificial valve configured to replace or repair a heart valve such as the aortic valve.

29. The endoluminal prosthesis of claim 24 wherein said first structure is configured to provide stabilisation of the second structure.

30. The endoluminal prosthesis of claim 24 wherein at least one connector comprises first and second portions that are connected to the first and second structures, respectively, and wherein the first and second portions are relatively moveable.

31. The endoluminal prosthesis of claim 30 wherein the first and second portions are slidably connected relative to each other.

32. The endoluminal prosthesis of claim 24 wherein at least one of said plurality of connectors is a unitary structure.

33. The endoluminal prosthesis of claim 32 wherein said at least one connector comprises a curved and/or bent region and wherein the degree of curvature and/or bending of the curved and/or bent region is adjustable to vary the length of the connector between the first and second structures.

34. The endoluminal prosthesis of claim 24 wherein said connectors are connected to the first and/or second structures via respective flexible or articulating joints.

35. The endoluminal prosthesis of claim 24 wherein said first expandable structure is connected to a fourth structure, said fourth structure comprising a radially expandable stent.

36. The endoluminal prosthesis of claim 35 wherein said fourth structure comprises a radial force dampener.

37. The endoluminal prosthesis of claim 24, further comprising a locking mechanism to lock the length of the at least one of the plurality of connectors.

38. The endoluminal prosthesis of claim 24, wherein the length of at least one of the plurality of connectors is fixable, by deformation of the connector.

39. The endoluminal prosthesis of claim 24, wherein at least one connector of the plurality of connectors comprises first and second portions and wherein the first and second portions are relatively slidable to adjust the length of the connector.

40. The endoluminal prosthesis of claim 39, wherein the first portion of the at least one connector includes a first arm and the second portion includes a second arm, the second arm including a hub region with an orifice therethrough to slidably receive at least a portion of the first arm.

41. The endoluminal prosthesis of claim 40, wherein the first arm is slidably moveable relative to the second arm through the orifice, the first arm including a stop to prevent the first arm passing fully though the orifice and disengaging from the second arm.

42. The endoluminal prosthesis of claim 41, wherein at least part of the first arm includes a deformable portion, which upon deformation, locks the first arm relative to the second arm to prevent further slidable movement and to substantially lock the length of the at least one connector.

43. The endoluminal prosthesis of claim 24 wherein the lengths of the plurality of connectors change through changing of the relative positioning of the first and second expandable structures.

44. An endoluminal prosthesis for deployment in a body, comprising:
a first expandable structure,
a second expandable structure; and
a third structure connected between the first and second structures,
the third structure having an adjustable shape such as to permit displacement of one of the first and second structures relative to the body independently of displacement of the other of the first and second structures relative to the body.
